(19) United States
(12) Patent Application Publication
(10) Pub. No.: US 2024/0360160 A1
(43) Pub. Date: Oct. 31, 2024

(54) ALKYLALUMINOXANE COMPOSITIONS PREPARED FROM TRIMETHYLALUMINUM AND TRIETHYLALUMINUM AND USES THEREOF IN ETHYLENE OLIGOMERIZATION PROCESSES

(71) Applicant: Chevron Phillips Chemical Company LP, The Woodlands, TX (US)

(72) Inventors: Brooke L. Small, Kingwood, TX (US); Julie A. Leseberg, Kingwood, TX (US)

(21) Appl. No.: 18/642,883

(22) Filed: Apr. 23, 2024

Related U.S. Application Data
(60) Provisional application No. 63/498,537, filed on Apr. 27, 2023.

(51) Int. Cl. C07F 5/06; C07C 2/30

(52) U.S. Cl. CPC C07F 5/068; C07C 2/30; C07C 2531/14

(57) ABSTRACT

Alkylaluminoxane compositions are produced by a process that includes the steps of reacting trimethylaluminum, triethylaluminum, and water in a hydrocarbon solvent to form an alkylaluminoxane, and then removing insoluble aluminum-containing materials from the solvent to form the alkylaluminoxane composition containing from 0.1 to 20 wt. % of aluminum. Generally, the molar ratio of trimethylaluminum:triethylaluminum is from 5:95 to 80:20, and the molar ratio of water:aluminum is from 0.2:1 to 1:1. The alkylaluminoxane compositions can be utilized as an activator in transition metal-based catalyst systems and in ethylene oligomerization processes.

### REFERENCE TO RELATED APPLICATION

This application claims the benefit of U.S. Provisional Patent Application No. 63/498,537, filed on Apr. 27, 2023, the disclosure of which is incorporated herein by reference in its entirety.

### FIELD OF THE INVENTION

The present disclosure relates generally to alkylaluminoxane compositions and to processes for preparing alkylaluminoxane compositions utilizing a combination of trimethylaluminum (TMA) and triethylaluminum (TEA).

### BACKGROUND OF THE INVENTION

Aluminoxanes such as methylaluminoxane (MAO) are widely used activators in transition metal based catalyst systems. MAO is very expensive due in part to the required trimethylaluminum (TMA) reactant. Modified MAO (MMAO) materials, therefore, are available in which a portion of the TMA reactant is replaced with triisobutylaluminum (TIBA). However, there is a continued need for improved activators with a combination of acceptable catalytic activation potential, shelf-life and catalyst stability, solubility in non-aromatic hydrocarbons, and cost effectiveness. It is to these ends that the present invention is generally directed.

### SUMMARY OF THE INVENTION

This summary is provided to introduce a selection of concepts in a simplified form that are further described herein. This summary is not intended to identify required or essential features of the claimed subject matter. Nor is this summary intended to be used to limit the scope of the claimed subject matter.

Alkylaluminoxane compositions and processes for preparing the alkylaluminoxane compositions are described herein. In one aspect, for instance, the alkylaluminoxane composition can comprise (i) an alkylaluminoxane having random repeating units of formula (A) and formula (B), and (ii) a hydrocarbon solvent, and the amount of aluminum in the composition can range from 0.1 to 20 wt. %. In formulas (A) and (B), R is methyl and R¹ is ethyl at a molar ratio of methyl:ethyl from 5:95 to 80:20:

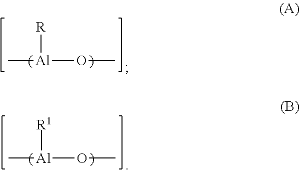

Another alkylaluminoxane composition provided herein can be produced by a process comprising (a) reacting trimethylaluminum (TMA), triethylaluminum (TEA), and water in a hydrocarbon solvent to form an alkylaluminoxane, and (b) removing insoluble aluminum-containing materials from the solvent to form the alkylaluminoxane composition containing from 0.1 to 20 wt. % of aluminum. In step (a), the molar ratio of TMA:TEA is from 5:95 to 80:20, and the molar ratio of water:Al is from 0.2:1 to 1:1.

Also described herein are processes for preparing an alkylaluminoxane composition. A representative process can comprise (a) reacting trimethylaluminum (TMA), triethylaluminum (TEA), and water in a hydrocarbon solvent to form an alkylaluminoxane, and (b) removing insoluble aluminum-containing materials from the solvent to form the alkylaluminoxane composition containing from 0.1 to 20 wt. % of aluminum. In step (a), the molar ratio of TMA:TEA is from 5:95 to 80:20, and the molar ratio of water:Al is from 0.2:1 to 1:1.

The alkylaluminoxane compositions can be utilized as an activator and combined with a heteroatomic ligand transition metal compound complex (or a heteroatomic ligand and a transition metal compound) to prepare a catalyst composition, and while not limited thereto, the catalyst composition subsequently can be used in an ethylene oligomerization process to produce ethylene-based oligomers, such as 1-hexene and 1-octene.

Both the foregoing summary and the following detailed description provide examples and are explanatory only. Accordingly, the foregoing summary and the following detailed description should not be considered to be restrictive. Further, features or variations can be provided in addition to those set forth herein. For example, certain aspects can be directed to various feature combinations and sub-combinations described in the detailed description.

### BRIEF DESCRIPTION OF THE FIGURES

FIG. 1 presents a plot of aluminum loss versus the molar ratio of water:aluminum for the experiments of Examples 1-12.

FIG. 2 presents a plot of catalyst activity versus catalyst storage time for the alkylaluminoxane composition and MMAO oligomerization experiments of Example 13.

FIG. 3 presents a plot of catalyst activity versus catalyst storage time for the alkylaluminoxane composition, alkylaluminoxane composition mixed with TIBA, and MMAO oligomerization experiments of Example 14.

FIG. 4 presents a plot of catalyst activity versus catalyst storage time for the alkylaluminoxane composition oligomerization experiments of Example 15.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter can be described such that, within particular aspects, a combination of different features can be envisioned. For each and every aspect and/or feature disclosed herein, all combinations that do not detrimentally affect the designs, compositions, processes, and/or methods described herein are contemplated with or without explicit description of the particular combination. Additionally, unless explicitly recited otherwise, any aspect and/or feature disclosed herein can be combined to describe inventive features consistent with the present disclosure.

In this disclosure, while compositions and processes/methods are described in terms of “comprising” various materials or components and steps, the compositions and processes/methods also can “consist essentially of” or “consist of” the various materials or components and steps, unless stated otherwise. The terms “a,” “an,” and “the” are intended to include plural alternatives, e.g., at least one, unless otherwise specified.

Generally, groups of elements are indicated using the numbering scheme indicated in the version of the periodic table of elements published in Chemical and Engineering News, 63 (5), 27, 1985. In some instances, a group of elements can be indicated using a common name assigned to the group; for example, alkali metals for Group 1 elements, alkaline earth metals for Group 2 elements, transition metals for Group 3-12 elements, and halogens or halides for Group 17 elements.

For any particular compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diasteromers, and other optical isomers (if there are any), whether in enantiomeric or racemic forms, as well as mixtures of stereoisomers, as would be recognized by a skilled artisan, unless otherwise specified. For example, a general reference to hexene (or hexenes) includes all linear or branched, acyclic or cyclic, hydrocarbon compounds having six carbon atoms and 1 carbon-carbon double bond; a general reference to pentane includes n-pentane, 2-methyl-butane, and 2,2-dimethylpropane; and a general reference to a butyl group includes an n-butyl group, a sec-butyl group, an iso-butyl group, and a t-butyl group.

The terms “contacting” and “combining” are used herein to describe compositions and processes/methods in which the materials are contacted or combined together in any order, in any manner, and for any length of time, unless otherwise specified. For example, the materials can be blended, mixed, slurried, dissolved, reacted, treated, impregnated, compounded, or otherwise contacted or combined in some other manner or by any suitable method or technique.

The term “hydrocarbon” whenever used in this specification and claims refers to a compound containing only carbon and hydrogen. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon).

The term “oligomer” refers to a compound that contains from 2 to 20 monomer units. The terms “oligomerization product” and “oligomer product” include all products made by the “oligomerization” process, including the “oligomers” and products which are not “oligomers” (e.g., products which contain more than 20 monomer units, or solid polymer), but exclude other non-oligomer components of an oligomerization reactor effluent stream, such as unreacted ethylene, organic reaction medium, and hydrogen, amongst other components.

The terms “catalyst composition,” “catalyst mixture,” “catalyst system,” and the like, do not depend upon the actual product or composition resulting from the contact or reaction of the initial components of the disclosed or claimed catalyst composition/mixture/system, the nature of the active catalytic site, or the fate of the alkylaluminoxane and the heteroatomic ligand transition metal compound complex (or the alkylaluminoxane and the heteroatomic ligand and the transition metal compound) after combining these components. Therefore, the terms “catalyst composition,” “catalyst mixture,” “catalyst system,” and the like, encompass the initial starting components of the composition, as well as whatever product(s) may result from contacting these initial starting components. The terms “catalyst composition,” “catalyst mixture,” “catalyst system,” and the like, may be used interchangeably throughout this disclosure.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, the molar ratio of water:aluminum can be in various ranges. By a disclosure that the molar ratio of water:Al can range from 0.2:1 to 1:1, the intent is to recite that the molar ratio can be any ratio within the range and, for example, can include any range or combination of ranges from 0.2:1 to 1:1, such as from 0.2:1 to 0.8:1, from 0.3:1 to 0.8:1, from 0.3:1 to 0.7:1, from 0.3:1 to 0.6:1, from 0.4:1 to 0.8:1, from 0.4:1 to 0.6:1, from 0.4:1 to 0.5:1, or from 0.5:1 to 0.6:1, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods and materials similar or equivalent to those described herein can be used in the practice or testing of the invention, the typical methods and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION OF THE INVENTION

Disclosed herein are alkylaluminoxane compositions, processes for preparing the alkylaluminoxane compositions, catalyst compositions containing the alkylaluminoxane compositions and methods of making the catalyst compositions, and ethylene oligomerization processes utilizing the catalyst compositions. The alkylaluminoxane compositions are prepared from a mixture or combination of trimethylaluminum (TMA) and triethylaluminum (TEA).

An objective of this invention is to produce an alkylaluminoxane composition that is more cost effective than traditional MAO and MMAO activators. Another objective is to produce an alkylaluminoxane composition with solubility in non-aromatic hydrocarbons, with excellent shelf-life and stability in solution form, and with acceptable activating potential for certain transition metal catalysts. Unexpectedly, the alkylaluminoxane compositions prepared herein from a mixture or combination of trimethylaluminum (TMA) and triethylaluminum (TEA) meet these objectives.

### Alkylaluminoxane Compositions

In one aspect, an alkylaluminoxane composition is disclosed herein. This alkylaluminoxane composition can comprise (i) an alkylaluminoxane having random repeating units of formula (A) and formula (B), and (ii) a hydrocarbon solvent, and the amount of aluminum in the composition can range from 0.1 to 20 wt. %. In formulas (A) and (B), R is methyl and R¹ is ethyl at a molar ratio of methyl:ethyl in a range from 5:95 to 80:20:

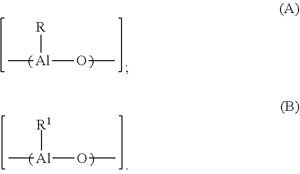

The total number of repeating units-inclusive of both (A) and (B)—in the alkylaluminoxane is not particularly limited, but often ranges from 2 to 20. Other typical ranges for the total number of repeating units can include from 3 to 18, from 5 to 20, from 5 to 18, from 6 to 20, from 6 to 15, from 8 to 20, or from 8 to 16, and the like.

The alkylaluminoxane having random repeating units of formula (A) and formula (B) also encompasses structures that may have cross-linked or aggregated units resulting in non-linear 2D and 3D alkylaluminoxane structures including cluster/cage structures, such as described in Collins, Chem. Eur. J. 2021, 27, 15460-71, and references therein.

In another aspect, an alkylaluminoxane composition is disclosed herein in which the composition is produced by a process that comprises (a) reacting trimethylaluminum (TMA), triethylaluminum (TEA), and water in a hydrocarbon solvent to form an alkylaluminoxane, and (b) removing insoluble aluminum-containing materials from the solvent to form the alkylaluminoxane composition containing from 0.1 to 20 wt. % of aluminum. In step (a), the molar ratio of TMA:TEA is from 5:95 to 80:20, and the molar ratio of water:Al is from 0.2:1 to 1:1.

In yet another aspect, a process for preparing an alkylaluminoxane composition is disclosed herein. This process can comprise (a) reacting trimethylaluminum (TMA), triethylaluminum (TEA), and water in a hydrocarbon solvent to form an alkylaluminoxane, and (b) removing insoluble aluminum-containing materials from the solvent to form the alkylaluminoxane composition containing from 0.1 to 20 wt. % of aluminum. In step (a), the molar ratio of TMA:TEA is from 5:95 to 80:20, and the molar ratio of water:Al is from 0.2:1 to 1:1.

Generally, the features of these compositions and processes (e.g., the relative amount of TMA to TEA (or methyl to ethyl), the hydrocarbon solvent, the amount of aluminum in the composition, and the relative amount of water to aluminum, among others) are independently described herein and these features can be combined without limitation, and in any combination to further describe the disclosed compositions and processes. Moreover, additional steps can be performed before, during, and/or after the steps of the processes, and can be utilized without limitation and in any combination to further describe the processes for preparing alkylaluminoxane compositions, unless stated otherwise. Likewise, the alkylaluminoxane compositions can contain other materials or components, unless stated otherwise.

The alkylaluminoxane compositions-which are generally solutions of the alkylaluminoxane in the hydrocarbon solvent—can contain from 0.1 to 20 wt. % of aluminum. For example, the composition can contain from 1 to 20 wt. % aluminum in one aspect, from 2 to 15 wt. % aluminum in another aspect, from 3 to 12 wt. % aluminum in another aspect, from 3 to 7 wt. % aluminum in another aspect, from 4 to 12 wt. % aluminum in yet another aspect, and from 5 to 10 wt. % aluminum in still another aspect. These weight percentages are based on the weight of the aluminum (in any form) in the composition as compared to the total weight of the composition. The amount of aluminum in the alkylaluminoxane composition is determined by ICP analysis. Dilute alkylaluminoxane compositions also are contemplated herein, and such dilute compositions or solutions of the alkylaluminoxane in the hydrocarbon solvent can contain from 0.1 to 2 wt. % of aluminum, and more often, from 0.1 to 1.5 wt. %, from 0.1 to 1 wt. %, or from 0.2 to 0.8 wt. %, and the like, and the amount of aluminum can depend upon viscosity and pumping considerations during use of the alkylaluminoxane compositions.

The relative amounts of TMA:TEA or methyl:ethyl in the processes or alkylaluminoxane compositions are not particularly limited. Nonetheless, illustrative and non-limiting ranges include molar ratios of TMA:TEA (or molar ratios of methyl:ethyl) from 10:90 to 70:30, from 15:85 to 60:40, from 15:85 to 40:60, from 15:85 to 30:70, from 15:85 to 25:75, from 20:80 to 70:30, from 20:80 to 40:60, from 20:80 to 30:70, or from 20:80 to 25:75. Often, it can be beneficial for the amount of TEA (or ethyl) to be greater than that of TMA (or methyl). In such circumstances, the molar ratio of TMA:TEA (or the molar ratio of methyl:ethyl) can be from 15:85 to 40:60, from 15:85 to 30:70, from 15:85 to 25:75, from 20:80 to 40:60, from 20:80 to 30:70, or from 20:80 to 25:75, while not limited thereto.

The alkylaluminoxane compositions are generally substantially free of water (contain less than 1 wt. % water), due to the consumption of water in the process for producing the alkylaluminoxane composition. More often, these compositions contain less than 1000 ppm (by weight) water, less than 500 ppm water, or less than 100 ppm water. In addition, the alkylaluminoxane compositions also can contain TEA, or TMA, or both TEA and TMA—this is unreacted or free TEA and/or TMA that has not been consumed in the process for producing the alkylaluminoxane composition.

A variety of hydrocarbon solvents can be used in the alkylaluminoxane compositions and the processes for preparing the alkylaluminoxane compositions disclosed herein. For instance, the hydrocarbon solvent can comprise any suitable saturated aliphatic hydrocarbon, any suitable aromatic hydrocarbon, any suitable linear a-olefin, or any combination thereof.

The saturated aliphatic hydrocarbon can be a linear aliphatic hydrocarbon, a branched aliphatic hydrocarbon, or a cyclic aliphatic hydrocarbon, as well as combinations thereof. Thus, the hydrocarbon solvent can comprise a linear alkane, a branched alkane, a cyclic alkane, or a combination thereof. Illustrative examples of saturated aliphatic hydrocarbons that can be utilized as the solvent, either singly or in combination, include propane, butane (e.g., n-butane or isobutane), pentane (e.g., n-pentane, neopentane, cyclopentane, or isopentane), hexane, heptane, octane, cyclohexane, methyl cyclohexane, and the like, as well combinations thereof. In a particular aspect of this disclosure, the hydrocarbon solvent can comprise (or consist essentially of, or consist of) cyclohexane.

Additionally or alternatively, the hydrocarbon solvent can comprise an aromatic hydrocarbon, such as benzene, toluene, ethylbenzene, xylene, styrene, mesitylene, and the like. Combinations of two or more aromatic hydrocarbons can be utilized, if desired.

Illustrative examples of linear a-olefins that can be utilized as the hydrocarbon solvent, either singly or in combination, include 1-butene, 1-hexene, 1-octene, 1-decene, 1-dodecene, 1-tetradecene, and the like, as well as combinations thereof.

Generally, the alkylaluminoxane compositions described herein can contain at least 40 wt. % of the hydrocarbon solvent, and more often, at least 50 wt. %, at least 60 wt. %, at least 70 wt. %, at least 80 wt. %, or at least 85 wt. % of composition is the hydrocarbon solvent. These weight percentages are based on the weight of the hydrocarbon solvent(s) as compared to the total weight of the composition.

As disclosed herein, the alkylaluminoxane compositions can be a solution, such as at standard temperature and pressure (25° C. and 1 atm). This means that there is no visual precipitation (or minimal visual precipitation) of the alkylaluminoxane in the hydrocarbon solvent under standard conditions. Accordingly, the alkylaluminoxane composition can be a solution in an aromatic hydrocarbon solvent, such as toluene, or the alkylaluminoxane composition can be solution in a saturated aliphatic solvent, such as cyclohexane, or the alkylaluminoxane composition can be a solution in a linear a-olefin solvent, such as 1-hexene.

Beneficially, such alkylaluminoxane compositions are solutions at standard temperature and pressure (25° C. and 1 atm) that can be stable-no visual precipitation (or minimal visual precipitation) of the alkylaluminoxane in the hydrocarbon solvent—for at least 1 day, and in some aspects, for at least 3 days, for at least 7 days, for at least 10 days, or for at least 14 days.

Referring now to the processes for preparing alkylaluminoxane compositions and to alkylaluminoxane compositions produced by the processes, step (a) is directed to reacting trimethylaluminum (TMA), triethylaluminum (TEA), and water in a hydrocarbon solvent to form an alkylaluminoxane. The molar ratio options for TMA:TEA in step (a) are described hereinabove. The molar ratio of water:Al in step (a) can range from 0.2:1 to 1:1. More often, the molar ratio of water:Al can fall within the range of from 0.2:1 to 0.8:1, such as from 0.3:1 to 0.8:1, from 0.3:1 to 0.7:1, from 0.3:1 to 0.6:1, from 0.4:1 to 0.8:1, from 0.4:1 to 0.6:1, from 0.4:1 to 0.5:1, or from 0.5:1 to 0.6:1. While not limited thereto, molar ratios in the 0.3:1 to 0.8:1 or the 0.3:1 to 0.6:1 range, such as around 0.5:1, provide a good balance of catalytic activity and aluminum loss.

The order of addition of the components in step (a) is not particularly limited, but in one aspect, the TMA, TEA, and hydrocarbon solvent often can be combined first, followed by adding the water. In another aspect, TMA and the solvent can be combined first, followed by the water, and then TEA, while in yet another aspect, TEA and the solvent can be combined first, followed by the water, and then TMA.

Step (a) can be performed at any suitable temperature, but typically below the standard boiling point of the hydrocarbon solvent, and considering that the reaction in step (a) is exothermic. Representative temperature ranges include from 10° C. to 90° C., from 20° C. to 70° C., from 15° C. to 55° C., from 20° C. to 45° C., or from 20° C. to 30° C., although not limited thereto. In these and other aspects, these temperature ranges also are meant to encompass circumstances where step (a) is conducted at a series of different temperatures, instead of at a single fixed temperature, wherein at least one temperature falls within the respective ranges. The pressure at which step (a) is conducted is not particularly limited, but can be at an elevated pressure (e.g., from 5 psig to 100 psig), at atmospheric pressure, or at any suitable sub-atmospheric pressure. In some instances, step (a) is conducted at atmospheric pressure, eliminating the need for pressurized vessels and their associated cost and complexity. Step (a) can be performed for any suitable time period and the addition of water in step (a) can be take place over any suitable time period. Illustrative and non-limiting time periods (e.g., for the complete or slow addition of water) include a wide range of time periods, such as from 1 min to 10 hr, from 1 min to 6 hr, from 5 min to 6 hr, from 5 min to 2 hr, or from 15 min to 3 hr, but is not limited solely to these time periods. Other appropriate temperature, pressure, and time ranges are readily apparent from this disclosure.

It should be noted that a catalyst is not required in step (a) to form the alkylaluminoxane, and thus the alkylaluminoxane is generally formed in the substantial absence of a catalyst (i.e., less than 1 wt. % of a catalyst based on the total weight of the TMA, TEA, water, and hydrocarbon solvent in step (a)). For instance, there can be less than 1000 ppm (by weight) of the catalyst, less than 100 ppm of the catalyst, or less than 10 ppm of the catalyst, and more often, no catalyst is used as demonstrated by the examples that follow.

In step (b), insoluble aluminum-containing materials are removed from the solvent to form the alkylaluminoxane composition, which contains from 0.1 to 20 wt. % of aluminum. The step of removing the insoluble aluminum-containing materials from the solvent can include any suitable technique, e.g., draining, decanting, pressing, centrifuging, filtering, sedimenting, stripping, evaporating, drying, and the like, or any combination thereof, and the respective technique(s) can be performed once or more than once. Often, the insoluble aluminum-containing materials are removed from the solvent via filtration.

It is desired to minimize the amount of aluminum removed in step (b) based on total aluminum before step (b), such as to less than or equal to 40 wt. %, less than or equal to 30 wt. %, less than or equal to 20 wt. %, or less than or equal to 10 wt. %. However, the aluminum loss often must be balanced with the amount of water added, the molar ratio of TMA:TEA, and the resulting catalytic activity. Given these considerations, it is common for the amount of aluminum removed in step (b)-based on total aluminum before step (b)—to fall within a range from 10 to 50 wt. %, from 15 to 45 wt. %, from 5 to 30 wt. %, from 5 to 20 wt. %, or from 20 to 40 wt. %.

In aspects of this invention, the processes for preparing alkylaluminoxane compositions do not require a step of removing (e.g., via filtration) the insoluble aluminum-containing materials from the solvent to form the alkylaluminoxane composition. In these aspects, therefore, the process for preparing an alkylaluminoxane composition can comprise reacting trimethylaluminum (TMA), triethylaluminum (TEA), and water in a hydrocarbon solvent (any hydrocarbon solvent disclosed herein) to form the alkylaluminoxane composition (which contains insoluble aluminum-containing materials). The alkylaluminoxane composition contains from 0.1 to 20 wt. % of aluminum (or any amount of aluminum disclosed herein). The molar ratio of TMA:TEA is from 5:95 to 80:20 (or any molar ratio disclosed herein), and the molar ratio of water:Al is from 0.2:1 to 1:1 (or any molar ratio disclosed herein). While not limited thereto, the alkylaluminoxane composition can be prepared and then used (without removing the insoluble aluminum-containing materials) directly in any of the catalyst compositions and oligomerization processes disclosed herein.

### Catalyst Compositions and Oligomerization Processes

Also encompassed herein are catalyst compositions and processes for producing catalyst compositions. An illustrative catalyst composition can contain (I) any of the alkylaluminoxane compositions disclosed herein, and (II) a heteroatomic ligand transition metal compound complex, or a heteroatomic ligand and a transition metal compound. An illustrative process for producing the catalyst composition can comprise (A) performing any of the processes to produce the alkylaluminoxane composition disclosed herein, and (B) contacting the alkylaluminoxane composition with a heteroatomic ligand transition metal compound complex (or a heteroatomic ligand and a transition metal compound) to form the catalyst composition.

Since the alkylaluminoxane compositions disclosed herein contain a hydrocarbon solvent, the resulting catalyst compositions, therefore, also can contain a hydrocarbon solvent, e.g., any suitable saturated aliphatic hydrocarbon or aromatic hydrocarbon. Combinations or two or more hydrocarbon solvents can be present in the catalyst composition.

The components of the catalyst composition can be combined at any suitable temperature, such as from 0° C. to 90° C., from 20° C. to 70° C., from 15° C. to 55° C., from 20° C. to 45° C., or from 20° C. to 30° C. (room temperature can be conveniently used), although not limited thereto. The catalyst composition can be formed in the presence or absence of an olefin (e.g., the olefin to be oligomerized, such as ethylene). If the catalyst composition is formed in a reactor at the time of contacting the olefin, then the appropriate pressures and temperatures will be those that are typical of the oligomerization process, discussed further below.

The heteroatomic ligand transition metal compound complex or the transition metal compound in the catalyst composition can include any suitable metal (or metals), including for instance, chromium, iron, cobalt, vanadium, titanium, zirconium, hafnium, and the like, or any combination thereof. In an aspect, the transition metal-based catalyst system can comprise chromium; alternatively, iron; alternatively, cobalt; alternatively, vanadium; alternatively, titanium; alternatively, zirconium; or alternatively, hafnium. Molar ratios of Al:transition metal (for example, Al:Cr or Al:Fe) in the catalyst composition can range from 10:1 to 5,000:1, such as from 50:1 to 3,000:1, from 75:1 to 3,000:1, from 75:1 to 2,000:1, from 100:1 to 2,000:1, or from 100:1 to 1,000:1, and the like.

While not being limited to use with any particular type of catalyst system, the alkylaluminoxane compositions disclosed herein are particularly well suited for use in conjunction with a catalyst that comprises a heteroatomic ligand transition metal compound complex or a heteroatomic ligand and a transition metal compound. Thus, the alkylaluminoxane compositions can be used in a catalyst system that comprises (i) a heteroatomic ligand chromium (or iron) compound complex, or (ii) a heteroatomic ligand and a chromium (or iron) compound. Exemplary heteroatomic ligand transition metal compound complexes (or heteroatomic ligands and transition metal compounds) that are well suited for use with the disclosed alkylaluminoxane compositions include those described, for example, in U.S. Pat. Nos. 8,680,003, 8,865,610, 9,962,689, 10,493,422, 10,464,862, 10,435,336, and 11,267,909. Often, molar ratios of Al:ligand of the heteroatomic ligand in the catalyst composition can range from 10:1 to 5,000:1, and more often, from 50:1 to 3,000:1, from 75:1 to 3,000:1, from 75:1 to 2,000:1, from 100:1 to 2,000:1, or from 100:1 to 1,000:1, and the like. When both a heteroatomic ligand and a transition metal compound are present, the molar ratio of ligand:transition metal often ranges from 10:1 to 1:10, and more often, from 8:1 to 1:8, from 5:1 to 1:5, from 4:1 to 1:4, or from 2:1 to 1:2, and the like. In some aspects, the transition metal compound is present in a molar excess relative to the heteroatomic ligand, although this is not a requirement.

Beneficially, such catalyst compositions are stable at standard temperature and pressure (25° C. and 1 atm). In this regard, the relationship between catalyst age and productivity is generally constant for at least one day, at least 2 days, at least 3 days, or at least 5 days. For instance, the change in productivity over time (A productivity/A time) can be less than or equal to 20% in one day, or less or equal to 15% in one day, or less than or equal to 10% in one day. The A productivity is the productivity at time zero minus the productivity at the particular time interval (e.g., 1 day). The catalyst composition stability is demonstrated in the Examples that follow.

Also encompassed herein are oligomerization processes. For instance, an oligomerization process consistent with one aspect of this invention can comprise (1) performing any process for producing a catalyst composition utilizing an alkylaluminoxane composition disclosed herein, (2) contacting ethylene, the catalyst composition, an organic reaction medium, and optionally hydrogen, in an oligomerization reactor, (3) forming an oligomer product in the oligomerization reactor, the oligomer product comprising hexenes and octenes, and (4) discharging an effluent stream from the oligomerization reactor, the effluent stream comprising unreacted ethylene and the oligomer product. An oligomerization process consistent with another aspect of this invention can comprise (1) contacting ethylene, any catalyst composition comprising an alkylaluminoxane composition disclosed herein, an organic reaction medium, and optionally hydrogen, in an oligomerization reactor, (2) forming an oligomer product in the oligomerization reactor, the oligomer product comprising hexenes and octenes, and (3) discharging an effluent stream from the oligomerization reactor, the effluent stream comprising unreacted ethylene and the oligomer product.

Among other constituents, the effluent stream contains an oligomer product, which can comprise hexenes and octenes, as well as other C₄⁺ linear alpha olefins. The amount of octenes in the oligomer product typically can fall within a range from 20 to 99 wt. %, based on the total amount of oligomers in the oligomer product. In an aspect, the minimum amount of octenes in the oligomer product can be 20, 30, or 40 wt. %. In another aspect, the maximum amount of octenes in the oligomer product can be 99, 95, 92.5, 90, 87.5, or 85 wt. %. Generally, the amount of octenes in the oligomer product can range from any minimum amount of octenes in the oligomer product to any maximum amount of octenes in the oligomer product described herein. For instance, the amount of octenes-based on the total weight of oligomers in the oligomer product—can be from 30 to 95 wt. %, from 40 to 95 wt. %, from 40 to 90 wt. %, from 20 to 90 wt. %, from 30 to 87.5 wt. %, from 30 to 85 wt. %, from 40 to 87.5 wt. %, from 40 to 85 wt. %, from 20 to 60 wt. %, from 30 to 55 wt. %, or from 40 to 55 wt. % octenes.

Additionally or alternatively, the oligomer product can contain any suitable amount of hexenes. In an aspect, the minimum amount of hexenes in the oligomer product can be 15, 20, 25, 30, or 35 wt. %. In another aspect, the maximum amount of hexenes in the oligomer product can be 75, 65, 60, 55, or 50 wt. %. Generally, the amount of hexenes in the oligomer product can range from any minimum amount of hexenes in the oligomer product to any maximum amount of hexenes in the oligomer product described herein. For instance, the amount of hexenes-based on the total weight of oligomers in the oligomer product—can be from 20 to 60 wt. %, from 25 to 55 wt. %, or from 30 to 50 wt. % hexenes.

The amount of conversion of ethylene in the oligomerization reactor is not particularly limited, and generally the minimum ethylene conversion can be at least 20, 30, 35, 40, 45, or 50 wt. %, while the maximum ethylene conversion can be 99, 95, 90, 80, 75, 70, or 65 wt. %. Generally, the ethylene conversion in the reactor can range from any minimum conversion to any maximum conversion described herein. For instance, the ethylene conversion can range from 20 to 95 wt. %, from 30 to 90 wt. %, from 40 to 80 wt. %, from 50 to 70 wt. %, or from 55 to 65 wt. %. The ethylene conversion is based on the amount of ethylene entering the reactor and the amount of (unreacted) ethylene in the effluent stream.

Referring now to the step of contacting ethylene, the catalyst composition, the organic reaction medium, and optionally hydrogen, in the oligomerization reactor. Hydrogen use is optional in this step, thus in one aspect, hydrogen is not present in this step of the process, while in another aspect, hydrogen is present in this step of the process.

Ethylene, the catalyst composition, the organic reaction medium, and hydrogen can be combined in any order or sequence and introduced into the oligomerization reactor separately or in any combination. For instance, hydrogen and ethylene can be combined and fed to the reactor separately from the catalyst composition. This invention is not limited by the manner in which the respective feed streams are introduced into the reactor. In one aspect, for instance, the catalyst composition can be formed first and then introduced into the oligomerization reactor. In this aspect, (I) the alkylaluminoxane composition is contacted with (II) the heteroatomic ligand transition metal compound complex, or the heteroatomic ligand and the transition metal compound, prior to being introduced into the reactor. In another aspect, however, the catalyst composition can be formed in the oligomerization reactor. In this aspect, (I) the alkylaluminoxane composition and (II) the heteroatomic ligand transition metal compound complex (or the heteroatomic ligand and the transition metal compound) are introduced separately into the reactor, and the catalyst composition is formed in the reactor.

Any suitable organic reaction medium can be used in the disclosed oligomerization processes, such as a hydrocarbon. Illustrative hydrocarbons can include, for example, saturated aliphatic hydrocarbons, aromatic hydrocarbons, linear a-olefins, and the like, as well as combinations thereof. The organic reaction medium can be selected from the same materials as that for the hydrocarbon solvent in the alkylaluminoxane compositions and related processes for their preparation. Thus, the organic reaction medium can comprise any alkane or aromatic or a-olefin hydrocarbon disclosed herein, as well as any combination thereof. While not required, the organic reaction medium can comprise the same material as that of the hydrocarbon solvent. Further, in a particular aspect of this disclosure, the organic reaction medium can comprise (or consist essentially of, or consist of) cyclohexane.

Forming the oligomer product in the oligomerization reactor can be performed at any suitable oligomerization temperatures and pressure. Often, the oligomer product can be formed at a minimum temperature of 0° C., 20° C., 30° C., 40° C., 45° C., or 50° C.; additionally or alternatively, at a maximum temperature of 165° C., 160° C., 150° C., 140° C., 130° C., 115° C., 100° C., or 90° C. Generally, the oligomerization temperature at which the oligomer product is formed can be in a range from any minimum temperature disclosed herein to any maximum temperature disclosed herein. Accordingly, suitable non-limiting ranges can include the following: from 0 to 165, from 20 to 160, from 20 to 115, from 40 to 160, from 40 to 140, from 50 to 150, from 50 to 140, from 50 to 130, from 50 to 100, from 60 to 115, from 70 to 100, or from 75 to 95° C. Other appropriate oligomerization temperatures and temperature ranges are readily apparent from this disclosure.

The oligomer product can be formed at a minimum pressure (or ethylene partial pressure) of 50 psig (344 kPa), 100 psig (689 kPa), 200 psig (1.4 MPa), or 250 psig (1.5 MPa); additionally or alternatively, at a maximum pressure (or ethylene partial pressure) of 4,000 psig (27.6 MPa), 3,000 psig (20.9 MPa), 2,000 psig (13.8 MPa), or 1,500 psig (10.3 MPa). Generally, the oligomerization pressure (or ethylene partial pressure) at which the oligomer product is formed can be in a range from any minimum pressure disclosed herein to any maximum pressure disclosed herein. Accordingly, suitable non-limiting ranges can include the following: from 50 psig (344 kPa) to 4,000 psig (27.6 MPa), from 100 psig (689 kPa) to 3,000 psig (20.9 MPa), from 100 psig (689 kPa) to 2,000 psig (13.8 MPa), from 200 psig (1.4 MPa) to 2,000 psig (13.8 MPa), from 200 psig (1.4 MPa) to 1,500 psig (10.3 MPa), or from 250 psig (1.5 MPa) to 1,500 psig (10.3 MPa). Other appropriate oligomerization pressures (or ethylene partial pressures) are readily apparent from this disclosure.

When used, hydrogen can be fed directly to the reactor, or hydrogen can be combined with an ethylene feed prior to the reactor. In the reactor, the hydrogen partial pressure can be at least 1 psig (6.9 kPa), 5 psig (34 kPa), 10 psig (69 kPa), 25 psig (172 kPa), or 50 psig (345 kPa); additionally or alternatively, a maximum hydrogen partial pressure of 2000 psig (13.8 MPa), 1750 psig (12.1 MPa), 1500 psig (10.3 MPa), 1250 psig (8.6 MPa), 1000 psig (6.9 MPa), 750 psig (5.2 MPa), 500 psig (3.4 MPa), or 400 psig (2.8 MPa). Generally, the hydrogen partial pressure can range from any minimum hydrogen partial pressure disclosed herein to any maximum hydrogen partial pressure disclosed herein. Therefore, suitable non-limiting ranges for the hydrogen partial pressure can include the following ranges: from 1 psig (6.9 kPa) to 2000 psig (13.8 MPa), from 1 psig (6.9 kPa) to 1750 psig (12.1 MPa), from 5 psig (34 kPa) to 1500 psig (10.3 MPa), from 5 psig (34 kPa) to 1250 psig (8.6 MPa), from 10 psig (69 kPa) to 1000 psig (6.9 MPa), from 10 psig (69 kPa) to 750 psig (5.2 MPa), from 10 psig (69 kPa) to 500 psig (3.5 MPa), from 25 psig (172 kPa) to 750 psig (5.2 MPa), from 25 psig (172 kPa) to 500 psig (3.4 MPa), from 25 psig (172 kPa) to 400 psig (2.8 MPa), or from 50 psig (345 kPa) to 500 psig (3.4 MPa). Other appropriate hydrogen partial pressures in the reactor for the formation of the oligomer product are readily apparent from this disclosure.

The oligomerization reactor in which the oligomer product is formed can comprise any suitable reactor. Non-limiting examples of reactor types can include a stirred tank reactor, a plug flow reactor, or any combination thereof; alternatively, a fixed bed reactor, a continuous stirred tank reactor, a loop slurry reactor, a solution reactor, a tubular reactor, a recycle reactor, or any combination thereof. In an aspect, there can be more than one reactor in series or in parallel, and including any combination of reactor types and arrangements. Moreover, the oligomerization process used to form the oligomer product can be a continuous process or a batch process, or any reactor or vessel utilized in the process can be operated continuously or batchwise.

### EXAMPLES

The invention is further illustrated by the following examples, which are not to be construed in any way as imposing limitations to the scope of this invention. Various other aspects, modifications, and equivalents thereof which, after reading the description herein, can suggest themselves to one of ordinary skill in the art without departing from the spirit of the present invention or the scope of the appended claims.

The general procedure for producing alkylaluminoxane compositions was as follows. In a drybox, a stirbar was added to a three-necked 1-liter flask. The flask was fitted with a thermocouple on one opening, a vent line on another opening, and a third opening was used to add the reagents. At room temperature (˜23° C.), trimethylaluminum (TMA) and triethylaluminum (TEA), either neat or in aliphatic or aromatic diluents, respectively, were added to the flask, followed by additional diluent to target a final weight percent aluminum in the finished product. Stirring was begun, making sure that the tip of the thermocouple was submerged in the liquid, and a septum was placed on the third neck of the flask. The appropriate amount of water, targeting the desired water to aluminum ratio, was then slowly added by a syringe through the septum, with this addition resulting in immediate heat generation and venting of gases through the vent line. In a typical reaction, addition of the water required about one hour, but this amount of time depended on the size of the batch, the water:aluminum ratio, and the resultant exotherm. Reaction temperatures reached as high as 65° C., but no preferred maximum or minimum exotherm was targeted. Following the water addition, the reaction was allowed to continue stirring until reaching about 30° C., and the reaction mixture was filtered to remove precipitated species. The filtrate was then analyzed by ICP to determine the weight percent aluminum in the final product. By determining the percentage of aluminum in the final product as well as the mass of the aluminoxane solution, it was possible to calculate the percent of “lost” aluminum (insolubles) isolated as precipitate.

The ICP method involved taking a carefully weighed aliquot from the final product, quenching the alkyls with a carefully weighed amount of heavy alcohol, and weighing this combination (some losses occur during quenching). The weight percent aluminum of the quench mixture was then used to back-calculate the percent aluminum in the aluminoxane. It is not recommended to perform ICP experiments on toluene solutions, since the digestion step involves nitric acid. ICP analysis for the amount of aluminum (wt. %) in the composition and aluminum loss (wt. %) utilized a PerkinElmer Optima 8300 instrument.

### Examples 1-12

Examples 1-12 were conducted to determine the amount of aluminum loss, due to the formation of insoluble materials, as a function of the water:aluminum ratio. Table 1 summarizes the experiments of Examples 1-12. The reactions were initiated at room temperature, unless otherwise noted, and the reaction conditions shown in Table 1 include the relative molar amount of TEA:TMA and the hydrocarbon solvent utilized.

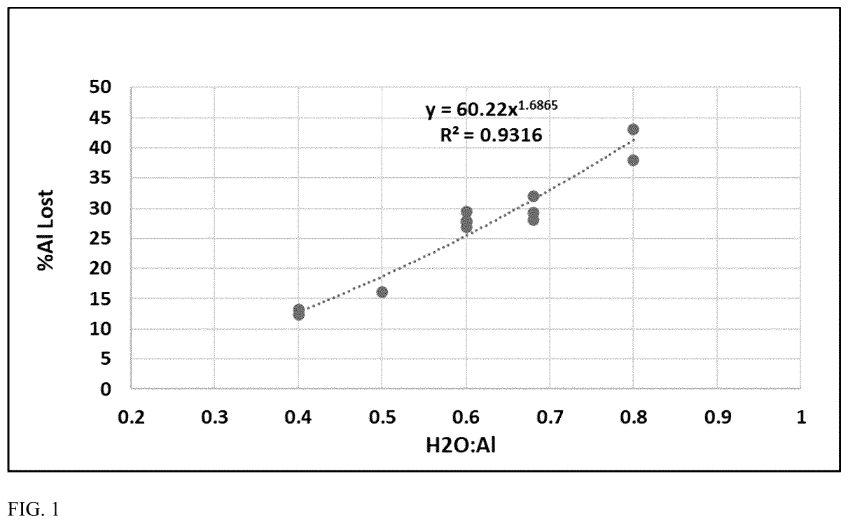

As demonstrated by FIG. 1 and the data in Table 1, aluminum loss generally increased with an increase in the molar ratio of water:aluminum. While aluminum loss might suggest that it would be better to use a lower water:aluminum ratio, this would need to be balanced with the overall activity of the resulting alkylaluminoxane composition. Also of note, any reduction in the amount of TMA (i.e., more TEA used to produce the alkylaluminoxane) also represents a cost savings. The alkylaluminoxane compositions of Examples 10-12 offer a particularly beneficial combination of properties, with Examples 11-12 providing lower amounts of aluminum loss.

TABLE 1

| | water: Al | Al Lost | Final Al | |
|---|---|---|---|---|
| Example | (mol) | (wt. %) | (wt. %) | Reaction Conditions |
| 1* | 0.8 | 43 | 1.22 | 50:50 TEA:TMA, toluene |
| 2* | 0.8 | 38 | 2.12 | 50:50 TEA:TMA, |
| | | | | toluene, 0° C. |
| 3* | 0.4 | 12 | 1.84 | 50:50 TEA:TMA, toluene |
| 4 | 0.6 | 28 | 3.38 | 75:25 TEA:TMA, xylene |
| 5 | 0.6 | 27 | 3.39 | 90:10 TEA:TMA, xylene |
| 6 | 0.6 | 28 | 3.37 | 75:25 TEA:TMA, xylene |
| 7 | 0.68 | 32 | 5.18 | 75:25 TEA:TMA, xylene |
| 8 | 0.68 | 28 | 4.74 | 75:25 TEA:TMA, |
| | | | | cyclohexane |
| 9 | 0.68 | 29 | 4.34 | 100 TEA, methyl |
| | | | | cyclohexane |
| 10 | 0.6 | 29 | 3.62 | 50:50 TEA:TMA, xylene |
| 11 | 0.5 | 16 | 5.70 | 75:25 TEA:TMA, xylene |
| 12 | 0.4 | 13 | 5.77 | 80:20 TEA:TMA, |
| | | | | cyclohexane |

*Aluminum loss determined by gravimetric or mass balance method. Other examples utilized ICP analysis to determine aluminum loss.

### Example 13

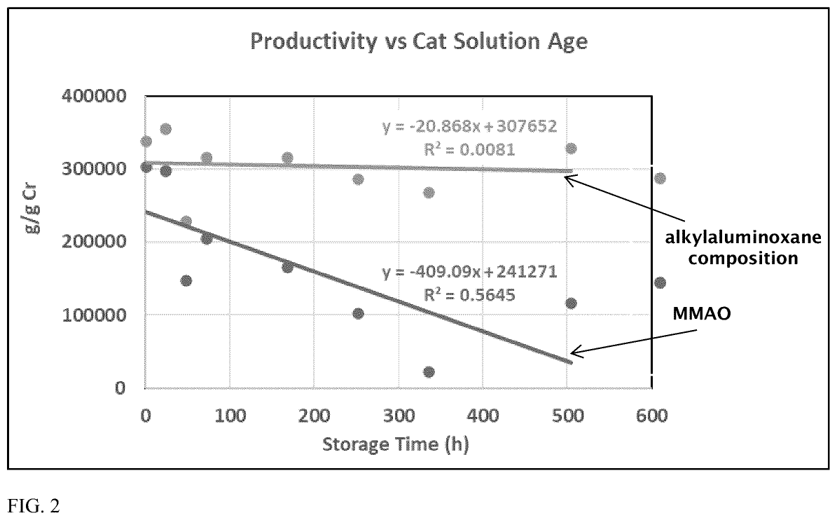

Example 13 demonstrates that catalyst compositions containing the alkylaluminoxane compositions described herein are more stable and have a longer shelf-life (constant oligomerization activity) as compared to analogous catalyst compositions containing MMAO. The results are summarized in FIG. 2.

Chromium catalyst compositions were prepared and ethylene oligomerizations were performed as follows. In a dry box, two glass scintillation vials were charged with equal amounts of a representative heteroatomic ligand chromium compound complex (a N²-phosphinyl guanidine chromium (III) trichloride tetrahydrofuran complex), 10.0 g of ethylbenzene, and 10.0 g of n-nonane (internal standard). After stirring for 30 minutes, MMAO at a 400:1 Al:Cr ratio was added to one of the vials (the control), and the inventive alkylaluminoxane composition of Example 4 (molar ratio of water:Al of 0.6:1, molar ratio of TEA:TMA of 75:25) was added to the second vial, also in a 400:1 Al:Cr ratio. Both vials were allowed to continue stirring for an additional hour, and the contents of each vial were diluted further with cyclohexane to give resultant solution concentrations of 2.0×10⁻⁴ M [Cr]. The solutions were stored in separate 500 mL bottles in the absence of air or moisture.

To test the stored catalyst solutions, 23.4 mL of each solution were pulled at different time intervals and diluted with cyclohexane to 200 mL total volume. The resultant solution was then charged to an evacuated 0.5 L stainless steel reactor heated to 70° C. Hydrogen (50 psig) was charged into the reactor followed by ethylene (875 psig). The reaction proceeded to exotherm to the target temperature of 85° C. with ethylene being fed on-demand to maintain the desired reactor pressure. After 30 min, the oligomerization reaction was rapidly cooled to 30° C., and then the unreacted ethylene and hydrogen gas were vented.

A liquid sample was collected, filtered, and analyzed by gas chromatography, typically using n-nonane as an internal standard, to determine the amount of oligomers produced, and therefore the catalyst activity in grams of oligomer product per gram of chromium. Both the control and the experimental catalyst solutions were tested at 1, 24, 48, and 72 hours, as well as 1, 1.5, 2, and 3 weeks. FIG. 2 shows no loss of productivity in the alkylaluminoxane composition catalyst solution, but significant productivity loss in the control catalyst solution.

The top (flat) line in FIG. 2 is from the series of oligomerization experiments utilizing the alkylaluminoxane composition and the bottom (decreasing) line is from the series of oligomerization experiments utilizing the comparative MMAO activator. Unexpectedly, catalyst compositions containing the alkylaluminoxane compositions disclosed herein had stable catalyst activity for 3 weeks; the catalyst had essentially the same activity for the entire three week test period. This is particularly beneficial in manufacturing operations, where large batches of the mixture can be prepared and stored for extended periods of time. In contrast, the catalyst composition containing the MMAO activator had a significant drop in activity after only 24 hr, and the activity was reduced in half after approximately 1 week.

### Example 14

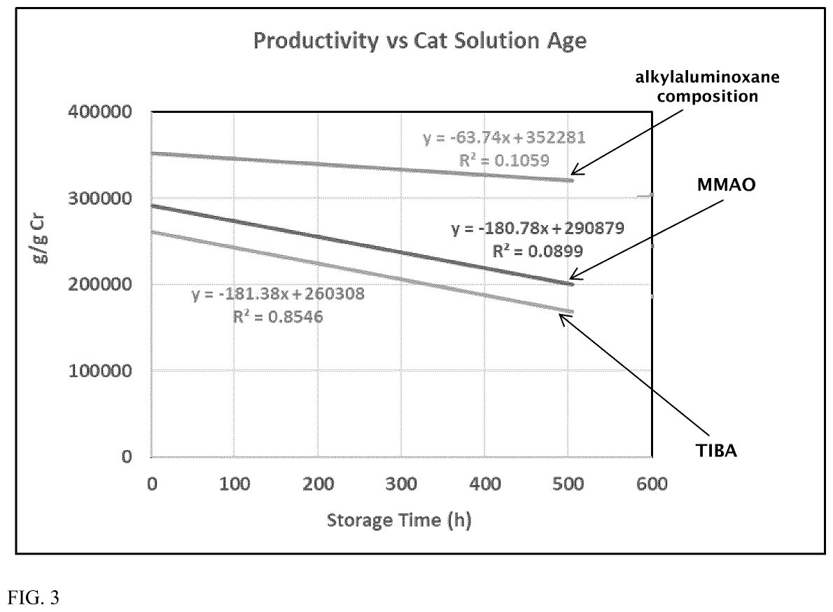

Example 14 demonstrates that catalyst compositions containing the alkylaluminoxane compositions described herein are more stable and have a longer shelf-life (constant oligomerization activity) as compared to analogous catalyst compositions containing MMAO or alkylaluminoxane compositions mixed with TIBA. The results are summarized in FIG. 3.

The experiments of Example 14 were performed similarly to that of Example 13, except the alkylaluminoxane of Example 6 (molar ratio of water:Al of 0.6:1, molar ratio of TEA:TMA of 75:25) was used to prepare the inventive catalyst sample. In addition, a third catalyst solution was prepared with extra TIBA added to the solution containing the chromium complex and the alkylaluminoxane of Example 6. For the experiment with TIBA, TIBA was added to the chromium complex and the alkylaluminoxane composition at a molar ratio of TIBA:Cr of 25:1. Similar to FIG. 2, FIG. 3 also demonstrates the unexpectedly superior stability and catalyst activity of catalyst compositions containing the alkylaluminoxane composition as compared to catalyst compositions containing the comparative MMAO activator. However, the catalyst compositions containing the alkylaluminoxane compositions mixed with TIBA performed worse than that of the inventive catalyst system, and similar to the control MMAO catalyst system. While not wishing to be bound by theory, it is believed that presence of TIBA caused the poor stability and catalyst activity of the catalyst composition containing the alkylaluminoxane composition mixed with TIBA.

### Example 15

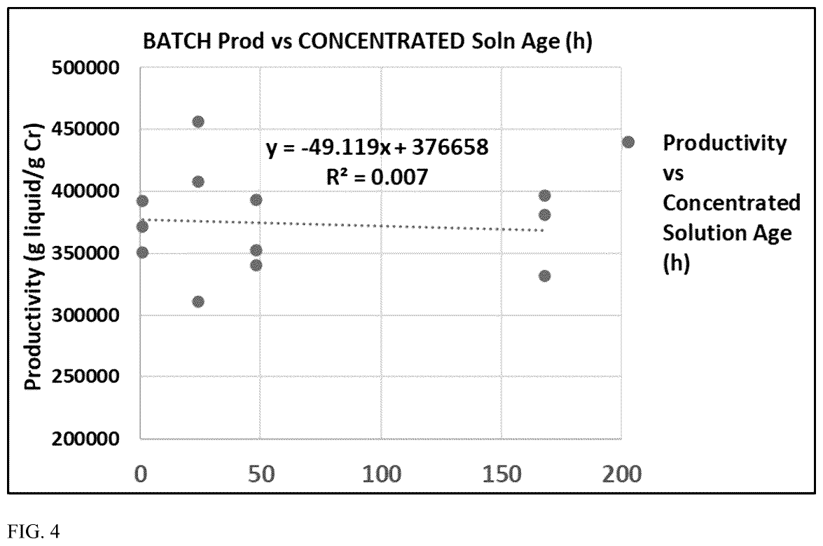

A third lifetime study was carried out, but only the inventive catalyst was prepared. The Cr complex was stirred and activated in the same manner as Example 13, except the alkylaluminoxane composition of Example 7 (molar ratio of water:Al of 0.68:1, molar ratio of TEA:TMA of 75:25) was used to activate the stirred suspension of the Cr complex. This activated catalyst solution was stored at this concentration. No control catalyst solution was prepared, and no additional cyclohexane was added to further dilute the solution to 2.0×10⁻⁴ M [Cr]. The resultant solution had a concentration of about 2.0×10⁻³ M [Cr]. Aliquots of the solution were tested for catalyst productivity at 1, 24, 48, and 168 hours. As demonstrated in FIG. 4, the triplicate run data had no loss in productivity after one week, which shows that even concentrated, activated catalyst solutions possess high stability for long periods of time.

### Examples 16-23

The heteroatomic ligand shown in Table 2 and a soluble iron source (transition metal compound) were combined in a small amount of an aromatic or aliphatic hydrocarbon solvent such as toluene, xylene, or cyclohexane, and added to a sealed NMR tube, which was then affixed to the impeller shaft of a high pressure autoclave, according to the procedure described in Organometallics 2003, 22, 3178 (Small). Cyclohexane solvent (200 ml) and cocatalyst were added to the sealed, evacuated autoclave, the reactor was pressurized with ethylene (400-800 psig range), and stirring was begun to break the glass and begin the reaction. Reaction initial (Tᵢₙᵢₜᵢₐₗ) and maximum (Tₘₐₓ) temperatures are shown in Table 3. Ethylene was fed on demand, and the reactions were terminated by degassing after 15 min. Products were analyzed by gas chromatography, using an internal standard.

The ethylene oligomerization experiments of Examples 16-23 are summarized in Table 3. Yields of volatile products (i.e. C₄) were extrapolated using the Schulz-Flory constant K, with total yields and productivities based on the C₄-C₂₆ products. In some cases, the Schulz-Flory constant is known to drift, typically upwards with increasing carbon number. Therefore, the extrapolation for calculating the K value for C₆/C₄ was based on the rate of change for the three prior fraction measurements. For example, K values for C₁₂/C₁₀, C₁₀/C₈, and Co/C₆ of 0.52, 0.50, and 0.48, respectively, would give an extrapolated K value of 0.46 for calculating the amount of 1-butene formed. As shown in Table 3, the alkylaluminoxane compositions of Examples 11-12 were excellent activators for the Fe-based catalyst systems of Examples 16-23.

TABLE 2

| | |
|---|---|
| 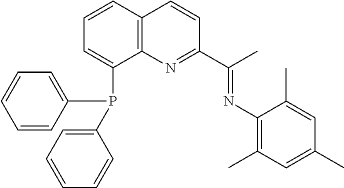 [SMILES: C/C(=N\c1c(C)cc(C)cc1C)c1ccc2cccc(P(c3ccccc3)c3ccccc3)c2n1] | 1 |
| 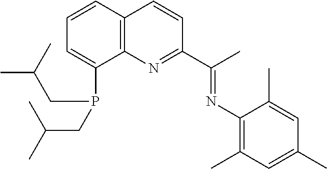 [SMILES: C/C(=N\c1c(C)cc(C)cc1C)c1ccc2cccc(P(CC(C)C)CC(C)C)c2n1] | 2 |
| 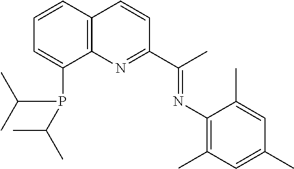 [SMILES: C/C(=N\c1c(C)cc(C)cc1C)c1ccc2cccc(P(C(C)C)C(C)C)c2n1] | 3 |
| 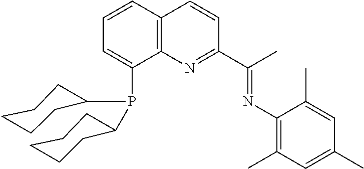 [SMILES: C/C(=N\c1c(C)cc(C)cc1C)c1ccc2cccc(P(C3CCCCC3)C3CCCCC3)c2n1] | 4 |
| 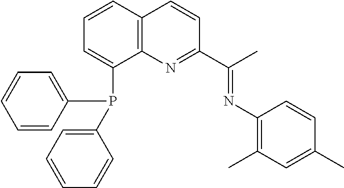 [SMILES: C/C(=N\c1ccc(C)cc1C)c1ccc2cccc(P(c3ccccc3)c3ccccc3)c2n1] | 5 |
| 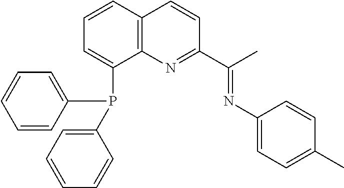 [SMILES: C/C(=N\c1ccc(C)cc1)c1ccc2cccc(P(c3ccccc3)c3ccccc3)c2n1] | 6 |
| 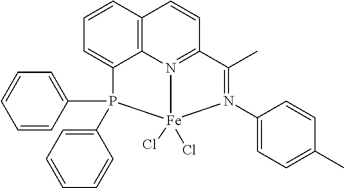 | 6•Fe |
| 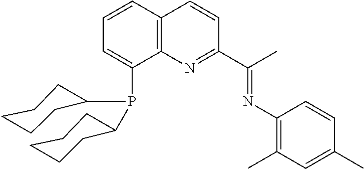 [SMILES: C/C(=N\c1ccc(C)cc1C)c1ccc2cccc(P(C3CCCCC3)C3CCCCC3)c2n1] | 7 |
| 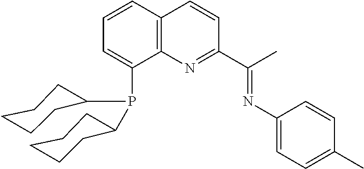 [SMILES: C/C(=N\c1ccc(C)cc1)c1ccc2cccc(P(C3CCCCC3)C3CCCCC3)c2n1] | 8 |
| 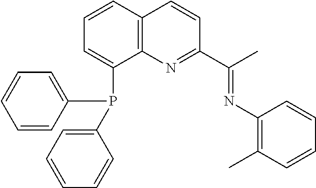 [SMILES: C/C(=N\c1ccccc1C)c1ccc2cccc(P(c3ccccc3)c3ccccc3)c2n1] | 9 |
| 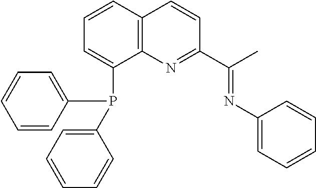 [SMILES: C/C(=N\c1ccccc1)c1ccc2cccc(P(c3ccccc3)c3ccccc3)c2n1] | 10 |

TABLE 3

| | | Ligand | Activator | | Tᵢₙᵢₜᵢₐₗ | K | K | Productivity | Yield |
|---|---|---|---|---|---|---|---|---|---|
| | Ligand | MW | Example | Metal source | Tₘₐₓ | (nC₁₂/ | (nC₁₀/ | (g/mmol | (g C₄- |
| Example | (mg) | (g/mol) | (Al:Ligand) | (metal:ligand) | (° C.) | nC₁₀) | nC₈) | ligand) | C₂₆) |
| 16 | 2 | 432 | 11 | Fe(octanoate)₃ | 30 | 0.770 | 0.747 | 4450 | 4.45 |
| | (0.43) | | (1000:1) | (4:1) | 50 | | | | |
| 17 | 3 | 404 | 11 | Fe(octanoate)₃ | 30 | 0.798 | 0.774 | 2310 | 2.31 |
| | (0.40) | | (1000:1) | (4:1) | 52 | | | | |
| 18 | 5 | 458 | 11 | Fe(octanoate)₃ | 30 | 0.458 | 0.428 | 32,300 | 32.3 |
| | (0.46) | | (1000:1) | (4:1) | 89 | | | | |
| *19 | 9 | 444 | 11 | Fe(octanoate)₃ | 75 | — | 0.437 | 40,460 | 44.8 |
| | (0.43) | | (1000:1) | (4:1) | 108 | | | | |
| 20 | 7 | 470 | 11 | Fe(octanoate)₃ | 30 | 0.472 | 0.447 | 14,600 | 14.6 |
| | (0.47) | | (1000:1) | (4:1) | 67 | | | | |
| 21 | 8 | 456 | 11 | Fe(octanoate)₃ | 30 | 0.200 | 0.151 | 35,700 | 35.7 |
| | (0.46) | | (1000:1) | (4:1) | 74 | | | | |
| 22 | 9 | 444 | 12 | Fe(octanoate)₃ | 30 | 0.508 | 0.500 | 22,300 | 22.3 |
| | (0.44) | | (1000:1) | (4:1) | 64 | | | | |
| 23 | 10 | 430 | 11 | Fe(octanoate)₃ | 30 | 0.345 | 0.213 | 21,400 | 21.4 |
| | (0.43) | | (1000:1) | (4:1) | 54 | | | | |

*Example 19 was run using 1-hexene as the diluent, and 96.8 wt. % purity 1-octene was still obtained, indicating that commercially relevant purities can be obtained under conditions of very high product (i.e., alpha-olefin) concentrations.

### Examples 24-25

Polymerization of 1-hexene using a metallocene catalyst and different activators was evaluated in Examples 24-25. A solution of bis (ethylcyclopentadienyl) zirconium (IV) dichloride (CAS No. 1291-32-3) was prepared from 2.0 mg of the metallocene complex per 1 ml of m-xylene. Two test reaction solutions were prepared using 0.5 mL of the standard metallocene solution (1.0 mg of metallocene) and 20 g of 1-hexene in each reaction vessel.

For Example 24, which served as the control, 0.45 g of a commercially available MMAO was added, the catalyst was activated, and polymerization began to occur, which was confirmed by a reaction exotherm that raised the reaction temperature from room temperature to 57° C. The Al:Zr molar ratio for this reaction was 400:1. The reaction was quenched after 2 hours, and the conversion was determined by GC to be 61%, by using the m-xylene as an internal standard.

For Example 25, an identical procedure was used, except the alkylaluminoxane composition of Example 7 (molar ratio of water:Al of 0.68:1, molar ratio of TEA:TMA of 75:25) was used instead of MMAO. For Example 25, no exotherm was observed following activation. The Al:Zr molar ratio for this reaction was 400:1. The reaction was quenched after 2 hours, and essentially no conversion was observed by GC. This surprising result shows that the alkylaluminoxanes of the invention are not effective for activation of metallocenes, and it was therefore unexpected that they are highly effective at activating Cr and Fe catalyst systems.

### Examples 26-29

Examples 26-29 were performed in substantially the same manner as that of Examples 1-12. Table 4 summarizes the experiments of Examples 26-29. The reactions were initiated at room temperature, and the reaction conditions shown in Table 4 include the molar ratio of TEA:TMA, the molar ratio of water:aluminum, and the hydrocarbon solvent utilized. Table 4 summarizes the final amount of aluminum in the composition and the amount of aluminum loss due to the formation of insoluble materials. In sum, each of Examples 26-29 produced an excellent alkylaluminoxane composition, with Examples 26-28 providing lower amounts of aluminum loss.

TABLE 4

| | water: Al | Al Lost | Final Al | |
|---|---|---|---|---|
| Example | (mol) | (wt. %) | (wt. %) | Reaction Conditions |
| 26 | 0.4 | 13 | 5.60 | 75:25 TEA:TMA, 1-hexene |
| 27 | 0.4 | 10 | 5.14 | 75:25 TEA:TMA, |
| | | | | cyclohexane |
| 28 | 0.5 | 7.7 | 5.56 | 75:25 TEA:TMA, |
| | | | | cyclohexane |
| 29 | 0.63 | 21 | 5.00 | 75:25 TEA:TMA, |
| | | | | cyclohexane |

* Aluminum loss determined by ICP analysis.

### Examples 30-32

Examples 30-32 were performed in substantially the same manner as that of Examples 16-23. Table 5 summarizes the ethylene oligomerization experiments of Examples 30-32, and reaction initial (Tᵢₙᵢₜᵢₐₗ) and maximum (Tₘₐₓ) temperatures are shown in Table 5. The structure of ligand 11 is shown below:

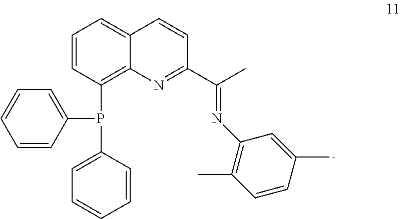 [SMILES: C/C(=N\c1cc(C)ccc1C)c1ccc2cccc(P(c3ccccc3)c3ccccc3)c2n1]

As shown in Table 5, the alkylaluminoxane compositions of Examples 27-29 were excellent activators for the Fe-based catalyst systems of Examples 30-32.

TABLE 5

| | | Ligand | Activator | | Tᵢₙᵢₜᵢₐₗ | K | K | Productivity | Yield |
|---|---|---|---|---|---|---|---|---|---|
| | Ligand | MW | Example | Metal source | Tₘₐₓ | (nC₁₂/ | (nC₁₀/ | (g/mmol | (g C₄- |
| Example | (mg) | (g/mol) | (Al:Ligand) | (metal:ligand) | (° C.) | nC₁₀) | nC₈) | ligand) | C₂₆) |
| 30 | 11 | 458 | 29 | Fe(octanoate)₃ | 30 | — | 0.493 | 61,290 | 60.6 |
| | (0.46) | | (1000:1) | (4:1) | 100 | | | | |
| 31 | 11 | 458 | 28 | Fe(octanoate)₃ | 30 | — | 0.468 | 53,010 | 53.2 |
| | (0.46) | | (1000:1) | (4:1) | 100 | | | | |
| 32 | 11 | 458 | 27 | Fe(octanoate)₃ | 30 | — | 0.512 | 35,710 | 35.9 |
| | (0.46) | | (1000:1) | (4:1) | 81 | | | | |

The invention is described herein with reference to numerous aspects and specific examples. Many variations will suggest themselves to those skilled in the art in light of the detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. An alkylaluminoxane composition comprising:
(i) an alkylaluminoxane having random repeating units of formula (A) and formula (B):

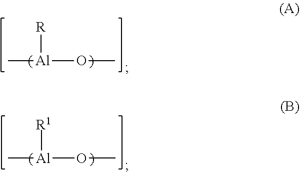

wherein:
R is methyl and R¹ is ethyl at a molar ratio of methyl:ethyl from 5:95 to 80:20; and
(ii) a hydrocarbon solvent;
wherein an amount of aluminum in the composition is from 0.1 to 20 wt. %.

Aspect 2. An alkylaluminoxane composition produced by a process comprising:
(a) reacting trimethylaluminum (TMA), triethylaluminum (TEA), and water in a hydrocarbon solvent to form an alkylaluminoxane, wherein:
a molar ratio of TMA:TEA is from 5:95 to 80:20; and
a molar ratio of water:Al is from 0.2:1 to 1:1; and
(b) removing insoluble aluminum-containing materials from the solvent to form the alkylaluminoxane composition containing from 0.1 to 20 wt. % of aluminum.

Aspect 3. A process for preparing an alkylaluminoxane composition, the process comprising:
(a) reacting trimethylaluminum (TMA), triethylaluminum (TEA), and water in a hydrocarbon solvent to form an alkylaluminoxane, wherein:
a molar ratio of TMA:TEA is from 5:95 to 80:20; and
a molar ratio of water:Al is from 0.2:1 to 1:1; and
(b) removing insoluble aluminum-containing materials from the solvent to form the alkylaluminoxane composition containing from 0.1 to 20 wt. % of aluminum.

Aspect 4. The composition or process defined in any one of aspects 1-3, wherein the alkylaluminoxane composition (e.g., solution) contains from 0.1 to 2 wt. %, from 0.1 to 1 wt. %, from 1 to 20 wt. %, from 2 to 15 wt. %, from 3 to 12 wt. %, from 3 to 7 wt. %, from 4 to 12 wt. %, or from 5 to 10 wt. % aluminum.

Aspect 5. The composition or process defined in any one of aspects 1-4, wherein the molar ratio of methyl:ethyl (or the molar ratio of TMA:TEA) is from 10:90 to 70:30, from 15:85 to 60:40, from 15:85 to 40:60, from 15:85 to 30:70, from 15:85 to 25:75, from 20:80 to 70:30, from 20:80 to 40:60, from 20:80 to 30:70, or from 20:80 to 25:75.

Aspect 6. The composition or process defined in any one of aspects 2-5, wherein the molar ratio of water:Al is from 0.2:1 to 0.8:1, from 0.3:1 to 0.8:1, from 0.3:1 to 0.7:1, from 0.3:1 to 0.6:1, from 0.4:1 to 0.8:1, from 0.4:1 to 0.6:1, from 0.4:1 to 0.5:1, or from 0.5:1 to 0.6:1.

Aspect 7. The composition or process defined in any one of aspects 1-6, wherein the composition is substantially free of water (less than 1 wt. % water), or the composition contains less than 1000 ppm (by weight) water, less than 500 ppm water, or less than 100 ppm water, and/or at least 40 wt. %, at least 50 wt. %, at least 60 wt. %, at least 70 wt. %, at least 80 wt. %, or at least 85 wt. % of the composition is the hydrocarbon solvent.

Aspect 8. The composition or process defined in any one of aspects 1-7, wherein the composition further comprises TEA, TMA, or both TEA and TMA (e.g., unreacted or free TEA and/or TMA).

Aspect 9. The composition or process defined in any one of aspects 2-8, wherein the TMA, TEA, and the hydrocarbon solvent are combined first, following by adding the water (and the water can be added over any suitable period of time).

Aspect 10. The composition or process defined in any one of aspects 2-9, wherein an amount of aluminum removed in step (b) based on total aluminum before step (b) is less than or equal to 40 wt. %, less than or equal to 30 wt. %, less than or equal to 20 wt. %, less than or equal to 10 wt. %, from 10 to 50 wt. %, from 15 to 45 wt. %, from 5 to 30 wt. %, from 5 to 20 wt. %, or from 20 to 40 wt. %.

Aspect 11. The composition or process defined in any one of aspects 2-10, wherein removing the insoluble aluminum-containing materials from the solvent comprises any suitable technique, e.g., draining, decanting, pressing, centrifuging, filtering, sedimenting, stripping, evaporating, drying, or any combination thereof, and performed once or more than once.

Aspect 12. The composition or process defined in any one of aspects 1-11, wherein the hydrocarbon solvent comprises a saturated aliphatic hydrocarbon, an aromatic hydrocarbon, a linear a-olefin, or any combination thereof.

Aspect 13. The composition or process defined in any one of aspects 1-11, wherein the hydrocarbon solvent comprises a saturated aliphatic hydrocarbon, e.g., propane, butane, pentane, hexane, heptane, octane, cyclohexane, methyl cyclohexane, or combinations thereof; or alternatively, the hydrocarbon solvent comprises cyclohexane.

Aspect 14. The composition or process defined in any one of aspects 1-11, wherein the hydrocarbon solvent comprises an aromatic hydrocarbon, e.g., benzene, toluene, xylene, cumene, ethylbenzene, or combinations thereof.

Aspect 15. The composition or process defined in any one of aspects 1-11, wherein the hydrocarbon solvent comprises a linear a-olefin, e.g., 1-butene, 1-hexene, 1-octene, 1-decene, 1-dodecene, 1-tetradecene, or combinations thereof.

Aspect 16. The composition or process defined in any one of aspects 1-15, wherein the composition is a solution at standard temperature and pressure (25° C. and 1 atm).

Aspect 17. The composition or process defined in aspect 16, wherein the solution is stable at standard temperature and pressure (25° C. and 1 atm) for at least 1 day, at least 3 days, at least 7 days, at least 10 days, or at least 14 days.

Aspect 18. The composition or process defined in any one of aspects 2-17, wherein the alkylaluminoxane is formed in the substantial absence of a catalyst.

Aspect 19. A process for producing a catalyst composition, the process comprising:
(A) performing the process defined in any one of aspects 3-18; and
(B) contacting the alkylaluminoxane composition with a heteroatomic ligand transition metal compound complex (or a heteroatomic ligand and a transition metal compound) to form the catalyst composition.

Aspect 20. A catalyst composition comprising:
(I) the alkylaluminoxane composition defined in any one of aspects 1-18; and
(II) a heteroatomic ligand transition metal compound complex (or a heteroatomic ligand and a transition metal compound).

Aspect 21. The process or composition defined in aspect 19 or 20, wherein the heteroatomic ligand transition metal compound complex and the alkylaluminoxane are present in the catalyst composition at a molar ratio of Al:transition metal (e.g., if the transition metal is chromium or iron, at a molar ratio of Al:Cr or Al:Fe), or the heteroatomic ligand and the alkylaluminoxane are present in the catalyst composition at a molar ratio of Al:ligand of the heteroatomic ligand, of from 10:1 to 5,000:1, from 50:1 to 3,000:1, from 75:1 to 3,000:1, from 75:1 to 2,000:1, from 100:1 to 2,000:1, or from 100:1 to 1,000:1.

Aspect 22. The process or composition defined in any one of aspects 19-21, wherein the catalyst composition comprises a heteroatomic ligand chromium (or iron) compound complex, or a heteroatomic ligand and a chromium (or iron) compound.

Aspect 23. The process or composition defined in any one of aspects 19-22, wherein the catalyst composition is stable at standard temperature and pressure (25° C. and 1 atm).

Aspect 24. An oligomerization process comprising:
(1) performing the process for producing a catalyst composition defined in any one of aspects 19-23;
(2) contacting ethylene, the catalyst composition, an organic reaction medium, and optionally hydrogen, in an oligomerization reactor;
(3) forming an oligomer product in the oligomerization reactor, the oligomer product comprising hexenes and octenes; and
(4) discharging an effluent stream from the oligomerization reactor, the effluent stream comprising unreacted ethylene and the oligomer product.

Aspect 25. An oligomerization process comprising:
(1) contacting ethylene, the catalyst composition defined in any one of aspects 20-23, an organic reaction medium, and optionally hydrogen, in an oligomerization reactor;
(2) forming an oligomer product in the oligomerization reactor, the oligomer product comprising hexenes and octenes; and
(3) discharging an effluent stream from the oligomerization reactor, the effluent stream comprising unreacted ethylene and the oligomer product.

Aspect 26. The oligomerization process defined in aspect 24 or 25, wherein the oligomer product comprises any amount of octenes disclosed herein, e.g., at least 20, 30 or 40 wt. %; a maximum of 99, 95, 92.5, 90, 87.5, or 85 wt. %; or from 20 to 99 wt. %, from 30 to 95 wt. %, from 40 to 95 wt. %, from 40 to 90 wt. %, from 20 to 90 wt. %, from 30 to 87.5 wt. %, from 30 to 85 wt. %, from 40 to 87.5 wt. %, from 40 to 85 wt. %, from 20 to 60 wt. %, from 30 to 55 wt. %, or from 40 to 55 wt. % octenes, based on the total amount of oligomers in the oligomer product.

Aspect 27. The oligomerization process defined in any one of aspects 24-26, wherein the oligomer product comprises any amount of hexenes disclosed herein, e.g., at least 15, 20, 25, 30, or 35 wt. %; a maximum of 75, 65, 60, 55, or 50 wt. %; or from 20 to 60 wt. %, from 25 to 55 wt. %, or from 30 to 50 wt. % hexenes, based on the total amount of oligomers in the oligomer product.

Aspect 28. The oligomerization process defined in any one of aspects 24-27, wherein the oligomerization reactor has any ethylene conversion disclosed herein, e.g., at least 20, 30, 35, 40, 45, or 50 wt. %; a maximum of 99, 95, 90, 80, 75, 70, or 65 wt. %; or from 20 to 95 wt. %, from 30 to 90 wt. %, from 40 to 80 wt. %, from 50 to 70 wt. %, or from 55 to 65 wt. % conversion, based on the amount of ethylene entering the reactor and the amount of ethylene in the effluent stream.

Aspect 29. The oligomerization process defined in any one of aspects 24-28, wherein hydrogen is contacted in the oligomerization reactor.

1. A process for preparing an alkylaluminoxane composition, the process comprising:
(a) reacting trimethylaluminum (TMA), triethylaluminum (TEA), and water in a hydrocarbon solvent to form an alkylaluminoxane, wherein:
a molar ratio of TMA:TEA is from 5:95 to 80:20; and
a molar ratio of water:Al is from 0.2:1 to 1:1; and
(b) removing insoluble aluminum-containing materials from the solvent to form the alkylaluminoxane composition containing from 0.1 to 20 wt. % of aluminum.

2. The process of claim 1, wherein:
the alkylaluminoxane composition contains from 2 to 15 wt. % aluminum;
the molar ratio of TMA:TEA is from 15:85 to 30:70; and
the hydrocarbon solvent comprises a saturated aliphatic hydrocarbon, an aromatic hydrocarbon, a linear a-olefin, or any combination thereof.

3. The process of claim 2, wherein the hydrocarbon solvent comprises cyclohexane.

4. The process of claim 1, wherein:
the composition is a solution at standard temperature and pressure; and
the solution is stable at standard temperature and pressure for at least 3 days.

5. The process of claim 1, wherein the molar ratio of water:Al is from 0.3:1 to 0.8:1.

6. The process of claim 1, wherein:
the composition is substantially free of water;
the composition further comprises TEA, TMA, or both TEA and TMA; and
the composition contains at least 50 wt. % of the hydrocarbon solvent.

7. The process of claim 1, wherein the TMA, TEA, and the hydrocarbon solvent are combined first, following by adding the water.

8. The process of claim 1, wherein an amount of aluminum removed in step (b) based on total aluminum before step (b) is from 15 to 45 wt. %.

9. A process for producing a catalyst composition, the process comprising:
(A) performing the process of claim 1; and
(B) contacting the alkylaluminoxane composition with a heteroatomic ligand transition metal compound complex or a heteroatomic ligand and a transition metal compound to form the catalyst composition.

10. The process of claim 9, wherein a molar ratio of Al:transition metal of the heteroatomic ligand transition metal compound complex or a molar ratio of Al:ligand of the heteroatomic ligand is in a range from 100:1 to 2,000:1.

11. The process of claim 10, wherein the catalyst composition is stable at standard temperature and pressure for at least 3 days.

12. The process of claim 9, wherein the catalyst composition comprises a heteroatomic ligand chromium compound complex, or a heteroatomic ligand and a chromium compound.

13. The process of claim 9, wherein the catalyst composition comprises a heteroatomic ligand iron compound complex, or a heteroatomic ligand and an iron compound.

14. An oligomerization process comprising:
(1) performing the process for producing a catalyst composition of claim 9;
(2) contacting ethylene, the catalyst composition, an organic reaction medium, and optionally hydrogen, in an oligomerization reactor;
(3) forming an oligomer product in the oligomerization reactor, the oligomer product comprising hexenes and octenes; and
(4) discharging an effluent stream from the oligomerization reactor, the effluent stream comprising unreacted ethylene and the oligomer product.

15. The oligomerization process of claim 14, wherein the catalyst composition is formed first and then introduced into the oligomerization reactor.

16. The oligomerization process of claim 14, wherein the catalyst composition is formed in the oligomerization reactor.

17. An alkylaluminoxane composition comprising:
(i) an alkylaluminoxane having random repeating units of formula (A) and formula (B): 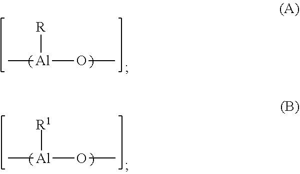
wherein:
R is methyl and R′ is ethyl at a molar ratio of methyl:ethyl from 5:95 to 80:20; and
(ii) a hydrocarbon solvent;
wherein an amount of aluminum in the composition is from 0.1 to 20 wt. %.

18. The composition of claim 17, wherein:
the alkylaluminoxane composition contains from 3 to 12 wt. % aluminum; and
the molar ratio of methyl:ethyl is from 15:85 to 30:70.

19. An alkylaluminoxane composition produced by a process comprising:
(a) reacting trimethylaluminum (TMA), triethylaluminum (TEA), and water in a hydrocarbon solvent to form an alkylaluminoxane, wherein:
a molar ratio of TMA:TEA is from 5:95 to 80:20; and
a molar ratio of water:Al is from 0.2:1 to 1:1; and
(b) removing insoluble aluminum-containing materials from the solvent to form the alkylaluminoxane composition containing from 0.1 to 20 wt. % of aluminum.

20. The composition of claim 19, wherein:
the alkylaluminoxane composition contains from 3 to 12 wt. % aluminum;
the molar ratio of TMA:TEA is from 15:85 to 30:70; and
the molar ratio of water:Al is from 0.3:1 to 0.8:1.
